(19) United States
(12) Patent Application Publication
(10) Pub. No.: US 2008/0299157 A1
(43) Pub. Date: Dec. 4, 2008

(54) SKIN CARE COMPOSITIONS HAVING UNIQUE TEXTURE PROPERTIES

(75) Inventors: Hani Fares, Somerset, NJ (US); John D. Strickland, Somerset, NJ (US); RitaMarie Guerrero, Morris Plains, NJ (US); Isabelle Hansenne, Westfield, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 12/112,375

(22) Filed: Apr. 30, 2008

Related U.S. Application Data
(60) Provisional application No. 60/941,341, filed on Jun. 1, 2007.

(51) Int. Cl. A61K 9/107; A61K 47/32; A61K 8/81; A61K 47/30; A61K 8/89

(52) U.S. Cl. 424/401; 514/772.4; 514/772.3

(57) ABSTRACT

The present invention is directed to a skin care composition comprising (a) at least one high internal phase emollient-in-water emulsion; (b) at least one thickening agent; (c) at least one primary emulsifier chosen from a water-in-silicone emulsifier; (d) at least one non-emulsifying silicone elastomer; (e) at least one active agent; and (f) water. Also disclosed is a method of treating skin with the above-disclosed skin care composition.

This application is based on and claims the benefit of U.S. Provisional Application Ser. No. 60/941,341, entitled SKIN CARE COMPOSITIONS HAVING UNIQUE TEXTURE PROPERTIES, filed Jun. 1, 2007, the entire disclosure of which is incorporated by reference herein.

### BACKGROUND OF THE INVENTION

There are many types of lotions, creams or gels available on the market which provide a benefit to the skin of the user. It is always the intention of the manufacturer to provide a product with functional and sensorial properties which appeal to the user. The products on the market available for application to the skin have excellent properties and, in general, their efficacy is obtained while also providing the user with textural and aromatic sensory experiences. Many of the ingredients usually associated with a good skin feel are formulated into emulsions of oil or silicone in water. The oil or silicone provides the slipperiness, or glide when applied to the skin. Silicone emulsions are more difficult to stabilize than conventional emulsions, but they provide a luxurious feel. However, the texture tends to be short and tends, in some cases, to be draggy. An opportunity exists to develop a product which provides skin benefits and a superior texture with a silky feel.

### BRIEF SUMMARY OF THE INVENTION

A first aspect of the present invention is directed to a skin care composition comprising (a) at least one high internal phase emollient-in-water emulsion; (b) at least one thickening agent; (c) at least one primary emulsifier chosen from a water-in-silicone emulsifier; (d) at least one non-emulsifying silicone elastomer; (e) at least one active agent; and (f) water, wherein the composition is a silicone-in-water emulsion.

A second aspect of the present invention is directed to a method of treating skin involving contacting the skin with the above-disclosed composition.

It has been surprisingly discovered that by using a water-in-silicone emulsifier in a silicone-in-water emulsion, a skin care composition is formed which delivers an exceptional silky feel to the skin of the user.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

High Internal Phase Emollient-in-Water Emulsion

In conventional emulsions, the dispersed phase contains spheres having a volume fraction of less than 0.74, which is the volume fraction of the most compact arrangement of spheres of equal radius. However, for high internal phase (HIP) emulsions, the dispersed phase may have a dispersed phase volume fraction as high as 0.99. The continuous phase, in such cases, forms a thin liquid film that separates polyhedral cells, and the HIP emulsion is stabilized by the adsorption of surfactant from the continuous phase onto the surface of the cells.

Methods of preparing HIP emulsions are described in U.S. Pat. No. 5,539,021 and U.S. Pat. No. 5,688,842, the entire contents of which are hereby incorporated by reference.

The emollient may be silicone based, non-silicone based or a combination thereof. Suitable silicone based emollients include, but are not limited to, non-volatile silicone oils such as dimethicone, volatile silicone oils such as cyclomethicone, chemically crosslinked siloxane polymers, also known as silicone elastomers, and mixtures thereof.

An example of a non-silicone based emollient used to form a HIP emulsion is petrolatum.

Preferred HIP emulsions for use in the composition of the present invention are those commercially available from Dow Corning under the tradenames DC 7-3099, DC 7-3100, DC 7-3101, DC 7-3110, DC 7-3111.

A particularly preferred HIP emulsion is DC 7-3101, commercially available from Dow Corning.

The HIP emulsions are generally present in the composition of the present invention in an amount ranging from about 10% to about 80% by weight; such as from about 35% to about 65% by weight; such as from about 40% to about 60% by weight, based on the total weight of the composition.

Thickening Agent

The thickening agent may be chosen from inorganic water thickeners, organic crystalline thickeners, polymeric thickeners, and mixtures thereof.

Suitable inorganic water thickeners for use in the composition of the present invention include, but are not limited to, silicas; clays such as a synthetic silicates (Laponite XLG and Laponite XLS from Southern Clay), and mixtures thereof; Acrylates/Vinyl Isodecanoate Crosspolymer (Stabylen 30 from 3V); Acrylates/C10-30 Alkyl Acrylate Crosspolymer (Pemulen TR1 and TR2); Carbomers (Aqua SF-1); hydrophobically modified sulfonic acid copolymers such as Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Ammonium Acryloyldimethyltaurate/Steareth-25 Methacrylate Crosspolymer (Aristoflex HMS); Acrylates/Ceteth-20 Itaconate Copolymer (Structure 3001 from National Starch); Polyacrylamides such as Sepigel 305 from SEPPIC; associative thickeners such as Aculyn 46 from Rohm and Haas and Acrylates/Aminoacrylates/C10-30 Alkyl PEG-20 Itaconate Copolymer (Structure Plus) from National Starch; cellulosic gels; hydroxypropyl starch phosphate such as Structure XL from National Starch; polyvinyl alcohol; and mixtures thereof.

Suitable organic crystalline thickeners for use in the composition of the present invention include, but are not limited to, ethylene glycol esters of fatty acids preferably having from about 16 to about 22 carbon atoms. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® available from Rheox, Inc. Other suitable thickeners are alkyl (C₁₆-C₂₂) dimethyl amide oxides such as stearyl dimethyl amine oxide. Also useful herein are long chain acyl derivatives, ethylene glycol esters of long chain carboxylic acids, long chain amine oxides, and alkanol amides of long chain carboxylic acids.

Suitable polymeric thickeners for use in the composition of the present invention include natural and modified natural polymers such as xanthan gum (Keltrol CG-T from CP Kelco), succinoglycan (Rheozan from Rhodia), gellan gum, pectin, alginates, starches including pregelatinized starches, modified starches, guars, cellulosic polymers, and mixtures thereof. These polymers may be neutral, anionic, cationic, zwitterionic, and/or amphoteric and include, but are not limited to, non-ionic guars (Jaguar HP105 & 120 available from Rhodia) and cationic guars (Jaguar Excel available from Rhodia), and mixtures thereof.

Preferred thickening agents include acrylate copolymers, such as those sold under the INCI denomination Acrylates/C10-30 Alkyl Acrylate Crosspolymer (commercially available as Pemulen TR1 and Pemulen TR2 from Noveon, Inc.), Carbomers (commercially available as Aqua SF-1 from Noveon, Inc.), and Ammonium Acryloyldimethyltaurate/Steareth-25 Methacrylate Crosspolymer (Aristoflex HMS from Clariant).

The thickening agent is generally present in the composition of the invention in an amount ranging from about 0.01% to about 5% by weight; such as from about 0.02% to about 3% by weight; such as from about 0.05% to about 2% by weight, based on the total weight of the composition.

Primary Emulsifier

The primary emulsifier employed in the composition of the present invention is a water-in-silicone emulsifier. The water-in silicone emulsifier is chosen from alkoxylated or polyglycerolated, linear or branched, polydimethylsiloxanes. The alkoxylation may be within the polymer or present as a graft. The water-in-silicone emulsifier has a low HLB value, such as below about 9, such as below about 7, such as below about 4.

Suitable water-in-silicone emulsifiers for use in the present invention include, but are not limited to, polyglycerin modified silicones such as Polyglyceryl-3 Disiloxane Dimethicone, commercially available as KF-6100 from the Shin-Etsu Company; ethoxylated/propoxylated silicones such as PEG/PPG-18/18 Dimethicone, commercially available as DC 5225C from the Dow Corning Company, and Cetyl PEG/PPG-10/1 Dimethicone commercially available as ABIL EM90 from Goldschmidt; ethoxylated silicones such as PEG-10 Dimethicone commercially available as KF-6017 from the Shin-Etsu Company; and mixtures thereof.

The water-in silicone emulsifier is generally present in the composition of the invention in an amount ranging from about 0.05% to about 20%, such as from about 0.1% to about 15%, such as from about 0.5% to about 10%.

Non-Emulsifying Silicone Elastomer

The composition of the present invention may contains at least one non-emulsifying silicone elastomer.

The term “non-emulsifying” defines elastomers not containing a hydrophilic chain, such as, for example, polyoxyalkylene or polyglycerol units.

The at least one non-emulsifying silicone elastomer, also known as elastomeric crosslinked organopolysiloxane may be conveyed in the form of a gel consisting of an elastomeric organopolysiloxane included in at least one hydrocarbon-based oil and/or one silicone oil. The elastomeric crosslinked organopolysiloxane may also be in powder form.

Suitable non-emulsifying silicone elastomers for use in the composition of the present invention include, but are not limited to, those sold under the names “DC 9040”, “DC 9041”, “DC 9509”, “DC 9505” and “DC 9506” by the company Dow Corning, and KSG-6, KSG-8, KSG-10, KSG-14, KSG-15, and KSG-16 by the company Shin-Etsu; SFE-168 and SFE-839 available from GE Silicones; Gransil SR-SYC available from Grant Industries; and mixtures thereof.

The non-emulsifying silicone elastomer may be present in the compositions of the present invention in an amount ranging from about 0.5% to about 80% by weight, such as from about 1% to about 60% by weight, such as from about 5% to about 15%, by weight based on the weight of the composition.

Active Agent

Any active agent capable of imparting a benefit onto skin may be employed in the present invention. It is to be understood that the active agents useful herein can in some instances provide more than one benefit or operate via more than one mode of action. Therefore, classifications herein are made for the sake of convenience and are not intended to limit the active to that particular application or applications listed.

Anti-Acne Active Agent

The compositions of the present invention may contain a safe and therapeutically effective amount of one or more pharmaceutically or cosmetically acceptable anti-acne active agents. Suitable anti-acne actives include, but are not limited to, resorcinol; sulfur; salicylic acid and salicylates; alpha-hydroxy acids; nonsteroidal anti-inflammatory agents; benzoyl peroxide; retinoic acid, isoretinoic acid and other retinoid compounds; adapalene; tazarotene; azelaic acid and azelaic acid derivatives; antibiotic agents, such as erythromycin and clyndamycin; zinc salts and complexes; and mixtures thereof.

Anti-Wrinkle Active Agent

The compositions of the present invention may contain a safe and effective amount of one or more anti-wrinkle active agents. Suitable anti-wrinkle active agents for use in the compositions of the present invention include, but are not limited to, sulfur-containing D and L amino acids and their derivatives and salts, particularly the N-acetyl derivatives; thiols; hydroxy acids (e.g., alpha-hydroxy acids such as lactic acid and glycolic acid and their derivatives and salts; or beta-hydroxy acids such as salicylic acid and salicylic acid salts and derivatives), urea and derivatives, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate); and mixtures thereof.

Anti-Oxidants/Radical Scavenger

The compositions of the present invention may contain a safe and effective amount of one or more anti-oxidant/radical scavengers. Suitable anti-oxidants/radical scavengers include, but are not limited to, ascorbic acid (vitamin C) and its salts; ascorbyl esters of fatty acids; ascorbic acid derivatives (e.g., magnesium ascorbyl phosphate, sodium ascorbyl phosphate, ascorbyl sorbate); tocopherol (vitamin E); tocopherol sorbate; tocopherol acetate; other esters of tocopherol; butylated hydroxy benzoic acids and their salts; 6-hydroxy-2,5,7,8-tetramethylchroman-2-carboxylic acid (commercially available under the tradename Trolox®); gallic acid and its alkyl esters, especially propyl gallate; uric acid and its salts and alkyl esters; sorbic acid and its salts; lipoic acid; amines (e.g., N,N-diethylhydroxylamine, amino-guanidine); sulfhydryl compounds (e.g., glutathione); dihydroxy fumaric acid and its salts; lysine pidolate; arginine pidolate; nordihydroguaiaretic acid; bioflavonoids; curcumin; lysine; methionine; proline; superoxide dismutase; silymarin; tea extracts; grape skin/seed extracts; melanin; rosemary extracts; and mixtures thereof.

Emollient

The composition of the present invention may contain one or more emollients. Suitable emollients include, but are not limited to, mineral oil; petrolatum; C₇-C₄₀ branched chain hydrocarbons; C₁-C₃₀ alcohol esters of C₁-C₃₀ carboxylic acids; C₁-C₃₀ alcohol esters of C₂-C₃₀ dicarboxylic acids; monoglycerides of C₁-C₃₀ carboxylic acids; diglycerides of C₁-C₃₀ carboxylic acids; triglycerides of C₁-C₃₀ carboxylic acids; ethylene glycol monoesters of C₁-C₃₀ carboxylic acids; ethylene glycol diesters Of C₁-C₃₀ carboxylic acids; propylene glycol monoesters of C₁-C₃₀ carboxylic acids; propylene glycol diesters of C₁-C₃₀ carboxylic acids; C₁-C₃₀ carboxylic acid monoesters and polyesters of sugars, for example, sefa cottonate (sucrose polycottonseedate); polydialkylsiloxanes; polydiarylsiloxanes; polyalkarylsiloxanes; cyclomethicones having 3 to 9 silicon atoms; vegetable oils; hydrogenated vegetable oils; polypropylene glycol C₄-C₂₀ alkyl ethers; di C₈-C₃₀ alkyl ethers; and mixtures thereof.

Self-Tanning Active Agent

The composition of the present invention may also contain one or more self-tanning active agents. Suitable self tanning agents include, but are not limited to, monocarbonyl or polycarbonyl compounds, such as isatin, alloxan, ninhydrin, glyceraldehyde, mesotartaric aldehyde, glutaraldehyde, erythrulose, dihydroxyacetone (DHA), and mixtures thereof.

In one or more embodiments of the present invention, the composition comprises a combination of a self-tanning agent and a sunscreen agent.

Skin Lightening/Whitening Agent

The composition of the present invention may contain one or more skin lightening/whitening agents. Suitable skin lightening/whitening agents include, but are not limited to, hydroquinone; azelaic acid and other related dicarboxylic acids, and salts and derivatives thereof; retinoids; kojic acid; arbutin; nicotinic acid and its precursors, salts and derivatives; arbutin; ascorbic acid and salts and derivatives thereof (e.g., magnesium ascorbyl phosphate or sodium ascorbyl phosphate); herbal extracts (e.g., licorice extract, mulberry extract); and mixtures thereof.

In one or more embodiments of the present invention, the composition comprises a combination of a skin lightening/whitening agent and a sunscreen agent.

Sunscreen Agent

The composition of the present invention may also contain one or more sunscreen agents, which are chemical absorbers that actually absorb harmful ultraviolet radiation, or physical blockers that block or reflect harmful ultraviolet radiation, or mixtures thereof. It is well known that chemical absorbers are classified, depending on the type of radiation they protect against, as either UV-A or UV-B absorbers. UV-A absorbers generally absorb radiation in the 320 to 400 nm region of the ultraviolet spectrum and UV-B absorbers generally absorb radiation in the 280 to 320 nm region of the ultraviolet spectrum.

Suitable chemical absorbers include, but are not limited to, p-aminobenzoic acid derivatives; anthranilates; benzophenones; camphor derivatives; cinnamic derivatives; dibenzoyl methanes (such as avobenzone also known as Parsol® 1789); diphenylacrylate derivatives; salicylic derivatives; triazine derivatives; benzimidazole compounds; bis-benzoazolyl derivatives; methylene bis-(hydroxyphenylbenzotriazole) compounds; sunscreen polymers; silicones; and mixtures thereof.

Suitable physical blockers include, but are not limited to, cerium oxides; chromium oxides; cobalt oxides; iron oxides; red petrolatum; silicone-treated titanium dioxide; titanium dioxide; zinc oxide; zirconium oxide; and mixtures thereof.

Exact amounts of the sunscreen agents will vary depending upon the sunscreen or sunscreens chosen and the desired Sun Protection Factor (SPF).

Humectant and Solute

The composition of the present invention may also contain one or more humectants and solutes which are non-volatile organic materials having a solubility of at least 5 parts in 10 parts water. Suitable water soluble, organic materials include, but are not limited to, polyols of the structure: R₁—O(CH₂—CR₂O)ₙH wherein R₁═H, C₁-C₄ alkyl; R₂═H, CH₃; and n=1-200; C₂-C₁₀ alkane diols; guanidine; glycolic acid and glycolate salts (e.g. ammonium and quaternary alkyl ammonium); lactic acid and lactate salts (e.g. ammonium and quaternary alkyl ammonium); polyhydroxy alcohols such as sorbitol, glycerol, hexanetriol, propylene glycol, hexylene glycol and the like; polyethylene glycol; sugars and starches and their derivatives such as alkoxylated glucose; panthenol (including D-, L-, and the D,L-forms); pyrrolidone carboxylic acid; hyaluronic acid and its salts; lactamide monoethanolamine; acetamide monoethanolamine; urea and derivatives; ethanol amines of the general structure (HOCH₂CH₂)ₓNH_{y} wherein x=1-3; y=0-2, and x+y=3; and mixtures thereof. Preferred polyols are chosen from glycerin, polyoxypropylene(1) glycerol and polyoxypropylene(3) glycerol, sorbitol, butylene glycol, propylene glycol, sucrose, urea, triethanolamine, and mixtures thereof.

Other Active Agent

The composition of the present invention may also contain other active agents. The International Cosmetic Ingredient Dictionary and Handbook (CTFA, Ninth Ed., 2002) describes a wide variety of nonlimiting cosmetic and skin care ingredients commonly used in the skin care industry, which is suitable for use in the compositions of the present invention. Suitable other active agents include, but are not limited to, abrasives; absorbents; antimicrobial and antifungal actives; aesthetic components such as fragrances, essential oils; astringents; binders; biological additives; buffering agents; bulking agents; chelating agents; chemical additives; denaturants; astringents; external analgesics; film formers or materials, e.g., polymers, for aiding the film-forming properties and substantivity of the composition; opacifying agents; pH adjusters; reducing agents; sequestrants; and mixtures thereof.

In general, the active agent will be present in the composition of the present invention in an amount of from about 0.01 to about 30% by weight, based on the total weight of the composition. It is to be understood, however, that the precise amount of active agent used will depend on the specific active agent(s) chosen and their intended effect on the skin.

Optional Ingredients

Emulsifiers

The composition of the present invention may also contain secondary emulsifiers. Suitable secondary emulsifiers that can be used according to the present invention include, but are not limited to, nonionic, cationic, anionic, and zwitterionic emulsifiers. Suitable secondary emulsifiers according to the present invention include, but are not limited to, acyl lactylates, alkyl phosphates, carboxylic acid copolymers, esters and ethers of glucose, esters of glycerin, esters of propylene glycol, esters of sorbitan anhydrides, esters of sorbitol, ethoxylated ethers, ethoxylated alcohols, fatty acid amides, fatty acid esters of polyethylene glycol, fatty esters of polypropylene glycol, polyoxyethylene fatty ether phosphates, soaps, alkoxylated polydimethylsiloxanes, and mixtures thereof.

Additional compounds suitable as secondary emulsifiers include, but are not limited to, emulsifying crosslinked siloxane elastomers such as Dimethicone/PEG-10/15 Crosspolymer commercially available from the Shin-Etsu Company as KSG-210, Dimethicone/Polyglycerin-3 Crosspolymer commercially available from the Shin-Etsu Company as KSG 710, Lauryl PEG-15 Dimethicone/Vinyl Dimethicone Crosspolymer commercially available from the Shin-Etsu Company as KSG-31, PEG-12 Dimethicone Crosspolymer, commercially available from the Dow-Corning Company as DC 9011. In one embodiment, the compound useful as a secondary emulsifier is Dimethicone/PEG-10/15 Crosspolymer.

For examples of other suitable secondary emulsifiers that can be used according to the present invention, see, for example, McCutcheon's, Detergents and Emulsifiers, North American Edition (2003), Allured Publishing Corporation, the entire contents of which are hereby incorporated by reference.

The secondary emulsifier may be present in the composition of the invention in an amount ranging from about 0.1 to about 10% by weight; such as from about 0.2 to about 7% by weight; such as from about 0.2 to about 5% by weight, based on the total weight of the composition.

Colorant

The composition of the present invention may also contain at least one cosmetically acceptable colorant such as a pigment or dyestuff. Suitable pigments include, but are not limited to, inorganic pigments; organic pigments; lakes; pearlescent pigments; irridescent or optically variable pigments; and mixtures thereof. A pigment should be understood to mean inorganic or organic, white or colored particles. The pigments may optionally be surface-treated within the scope of the present invention but are not limited to treatments such as silicones, perfluorinated compounds, lecithin, and amino acids.

Representative examples of inorganic pigments useful in the present invention include those chosen from rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); ferric blue (CI 77,510); and mixtures thereof.

Representative examples of organic pigments and lakes useful in the present invention include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on Cochineal Carmine (CI 75,570) and mixtures thereof.

Suitable pearlescent pigments useful in the present invention include those chosen from white pearlescent pigments such as mica coated with titanium oxide, mica coated with titanium dioxide, bismuth oxychloride, titanium oxychloride; colored pearlescent pigments such as titanium mica with iron oxides, titanium mica with ferric blue, chromium oxide and the like, titanium mica with an organic pigment of the above-mentioned type as well as those based on bismuth oxychloride; and mixtures thereof.

The precise amount and type of colorant employed in the compositions of the present invention will depend on the color, intensity and use of the composition and, as a result, will be determined by those skilled in the art of formulation.

Preservatives

The composition of the present invention may also contain at least one preservative. Suitable preservatives include, but are not limited to, alkyl para-hydroxybenzoates, wherein the alkyl radical has from 1, 2, 3, 4, 5 or 6 carbon atoms and preferably from 1 to 4 carbon atoms e.g., methyl para-hydroxybenzoate(methylparaben), ethyl para-hydroxybenzoate(ethylparaben), propyl para-hydroxybenzoate(propylparaben), butyl para-hydroxybenzoate(butylparaben) and isobutyl para-hydroxybenzoate(isobutylparaben). Other preservatives may also include phenoxyethanol, phenoxypropanol, phenoxyisopropanol, chlorhexidine, chlorhexidine gluconate, chlorhexidine hydrochloride. Mixtures of preservatives may certainly be used, e.g., the mixture of methyl-paraben, ethylparaben, propylparaben and butylparaben sold under the name Nipastat by Nipa, and the mixture of phenoxyethanol, methylparaben, ethylparaben, propylparaben and butylparaben sold under the name Phenonip, also by Nipa.

The preservatives may be present in the composition of the invention in an amount ranging from about 0.01% to about 10% by weight; such as from about 0.2% to about 5% by weight; such as from about 0.5% to about 3% by weight, based on the total weight of the composition.

Fillers

The compositions of the present invention may also contain at least one filler. Suitable fillers include, but are not limited to, silica powders; talc; polyamide particles and especially those sold under the name Orgasol by the company Atochem; polyethylene powders; microspheres based on acrylic copolymers, such as those based on ethylene glycol dimethacrylate/lauryl methacrylate copolymer sold by the company Dow Corning under the name Polytrap; expanded powders such as hollow microspheres and especially the microspheres commercially available from Kemanord Plast as Expancel or as Micropearl F 80 ED from Matsumoto; powders of natural organic materials such as crosslinked or noncrosslinked corn starch, wheat starch or rice starch, such as the powders of starch crosslinked with octenyl succinate anhydride, commercially available as Dry-Flo from National Starch; silicone resin microbeads commercially available as Tospearl from Toshiba Silicone; clays (bentone, laponite, saponite, etc.); and mixtures thereof.

The fillers may be present in the composition of the invention in an amount ranging from about 0.1% to about 50% by weight; such as from about 0.5% to about 30% by weight; such as from about 1% to about 20% by weight, based on the total weight of the composition.

Coated silicone elastomers

The composition may also contain coated silicone elastomers. Suitable coated silicone elastomers include, but are not limited to, silicone elastomer powders coated with microfine inorganic particles such as DC9701®, commercially available from Dow Corning, and silicone elastomer powders coated with a silicone resin, such as “KSP-100®”, “KSP-101®”, “KSP-102®”, “KSP-103®”, “KSP-104®”, “KSP-105®, “KSP-200®” and “KSP-300®”, available from Shin-Etsu.

The coated silicone elastomers may be present in the composition of the invention in an amount ranging from about 0.05% to about 20% by weight; such as from about 0.1% to about 15% by weight; such as from about 0.5% to about 10% by weight, based on the total weight of the composition.

The composition of the invention may be in the form of a treatment or makeup product for the skin. Examples thereof include, but are not limited to skin-care lotions, sunscreen/tanning products, and colored or uncolored cosmetic products.

### EXAMPLES

The present invention is further described in terms of the following non-limiting examples. Unless otherwise indicated, all parts and percentages are on a weight-by-weight percentage basis.

### Example 1

### Lotion

| Phase | Ingredient | % w/w |
|---|---|---|
| A | DI Water | 40.89 |
| | Sodium Hyaluronate | 1.00 |
| | Glycols | 9.00 |
| | Preservatives | 0.35 |
| | Ammonium Acryloyldimethyltaurate/Steareth- | 0.50 |
| | 25 Methacrylate Crosspolymer (Aristoflex HMS) | |
| B | Cyclopentasiloxane (and) Dimethicone | 40.00 |
| | Crosspolymer (and) Laureth-4 (and) Laureth-23 | |
| | (and) Dimethicone (DC 7-3101) | |
| | PEG-10 Dimethicone (KF-6017) | 0.50 |
| | Dimethicone (and) Dimethicone/Vinyl | 2.00 |
| | Dimethicone Crosspolymer (KSG-16) | |
| | Preservatives | 0.70 |
| C | Water | 5.00 |
| | Hydroxyethyl Urea | 0.01 |
| | Vitamin C | 0.02 |
| | Botanical Extracts | 0.03 |
| | TOTAL | 100.00 |

The above-disclosed lotion was found to exhibit an exceptionally silky feel and texture.

It is to be understood that the foregoing describes preferred embodiments of the invention and that modifications may be made therein without departing from the spirit or scope of the invention as set forth in the claims.

What is claimed is:

1. A skin care composition comprising:
(a) at least one high internal phase emollient-in-water emulsion;
(b) at least one thickening agent;
(c) at least one primary emulsifier chosen from a water-in-silicone emulsifier;
(d) at least one non-emulsifying silicone elastomer;
(e) at least one active agent; and
(f) water; and
wherein the composition is a silicone-in-water emulsion.

2. The composition of claim 1, wherein the emollient in (a) is silicone based.

3. The composition of claim 1, wherein (a) is present in an amount of from about 10% to about 80% by weight, based on the total weight of the composition.

4. The composition of claim 1, wherein (a) is present in an amount of from about 40% to about 60% by weight, based on the total weight of the composition.

5. The composition of claim 1, wherein (b) is present in an amount of about 0.01% to about 5% by weight, based on the total weight of the composition.

6. The composition of claim 1, wherein (b) is present in an amount of about 0.05% to about 2% by weight, based on the total weight of the composition.

7. The composition of claim 1, wherein (b) is chosen from acrylates, acrylate copolymers, carbomers, associative thickeners, and hydrophobically modified sulfonic acid copolymers.

8. The composition of claim 1, wherein (b) is Ammonium Acryloyldimethyltaurate/Steareth-25 Methacrylate Crosspolymer.

9. The composition of claim 1, wherein (c) is present in an amount of about 0.05% to about 20% by weight, based on the total weight of the composition.

10. The composition of claim 1, wherein (c) is present in an amount of about 0.5% to about 10% by weight, based on the total weight of the composition.

11. The composition of claim 1, wherein (c) is an alkoxylated polydimethylsiloxane.

12. The composition of claim 1, wherein (d) is present in an amount of about 0.5% to about 80%.

13. The composition of claim 1, wherein (d) is present in an amount of about 5% to about 15%.

14. The composition of claim 1, wherein (d) is a dimethicone/vinyl dimethicone crosspolymer.

15. A method of treating skin comprising contacting the skin with a skin care composition containing:
(a) at least one high internal phase emollient-in-water emulsion;
(b) at least one thickening agent;
(c) at least one primary emulsifier chosen from a water-in-silicone emulsifier;
(d) at least one non-emulsifying silicone elastomer;
(e) at least one active agent; and
(f) water; and
wherein the composition is a silicone-in-water emulsion.

16. The method of claim 15, wherein the emollient in (a) is silicone based.

17. The method of claim 15, wherein (a) is present in an amount of from about 10% to about 80% by weight, based on the total weight of the composition.

18. The method of claim 15, wherein (a) is present in an amount of from about 40% to about 60% by weight, based on the total weight of the composition.

19. The method of claim 15, wherein the (b) is present in an amount of about 0.01% to about 5% by weight, based on the total weight of the composition.

20. The method of claim 15, wherein (b) is present in an amount of about 0.05% to about 2% by weight, based on the total weight of the composition.

21. The method of claim 15, wherein (b) is chosen from acrylates, acrylate copolymers, carbomers, associative thickeners, and hydrophobically modified sulfonic acid copolymers.

22. The method of claim 15, wherein (b) is Ammonium Acryloyldimethyltaurate/Steareth-25 Methacrylate Crosspolymer.

23. The method of claim 15, wherein (c) is present in an amount of about 0.05% to about 20% by weight, based on the total weight of the composition.

24. The method of claim 15, wherein (c) is present in an amount of about 0.5% to about 10% by weight, based on the total weight of the composition.

25. The method of claim 15, wherein (c) is an alkoxylated polydimethylsiloxane.

26. The method of claim 15, wherein (d) is present in an amount of about 0.05% to about 20%.

27. The method of claim 15, wherein (d) is present in an amount of about 0.5% to about 15%.

28. The method of claim 15, wherein (d) is a dimethicone/vinyl dimethicone crosspolymer.
